(19) United States
(12) Patent Application Publication
(10) Pub. No.: US 2004/0072869 A1
(43) Pub. Date: Apr. 15, 2004

(54) Use of iloperidone

(76) Inventor: Hans O. Kalkman, Basel (CH)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/470,499

(22) PCT Filed: Feb. 4, 2002
(86) PCT No.: PCT/EP02/01130

(30) Foreign Application Priority Data
Feb. 5, 2001 (GB) 0102841.4

(51) Int. Cl. A61K 31/454

(52) U.S. Cl. 514/321

(57) ABSTRACT

The invention relates to the use of iloperidone in the treatment of affective disorders, including bipolar mood disorders.

The present invention relates to a new pharmaceutical use of 1-[4-[3-[4-(6-fluoro-1,2-benzisoxazol-3-yl)-1-piperidinyl]propoxy]-3-methoxyphenyl](iloperidone), and its pharmaceutically acceptable acid addition salts, hereinafter referred to as “agents of the invention”.

The agents of the invention and their production process are known e.g. from EP 402 644. This patent also discloses the use of the agents of the invention as antipsychotics.

In accordance with the present invention, it has now surprisingly been found that the agents of the invention are useful in the treatment of affective disorders including bipolar mood disorders.

The activity of the agents of the invention in said treatment is evidenced, for example, in the following tests suitable for detecting drugs having potential behavioral disinhibitory and/or sociotropic effects which are thought to be relevant for recovery from social withdrawal, a cardinal feature of depression and related psychiatric conditions.

a) The Half Enclosed Platform Test

This test is basically as described in Psychopharmacology, 1986, 89:31-37.

Groups of 12 male OF-1 mice are given vehicle or the substance 1 hour before being tested on the platform. The apparatus consists of a transparent platform perforated with 25 equally-spaced 1 cm holes. The platform is divided into equal halves by a 15 cm high, semi-rectangular wall enclosing one half of the platform, the other half having open edges. The whole platform rests on four 15 cm high legs. A line down the middle runs from the edge of one wall to the edge of the opposite wall. The experiment consists of placing a mouse on the midline and recording their behaviour for 5 minutes as they explore the platform. In particular, the mean frequencies and durations of the behavioral elements are recorded and statistical comparisons are determined using the Kruskal-Wallis “H” test followed by paired comparisons between control and treatment groups using the Mann-Whitney U-test. Probabilities (p=/<0.05) quoted are 2-tailed.

At doses of about 0.3 to about 10 mg/kg p.o., the agents of the invention significantly increase exploratory behaviour, such as stretched attend posture, head raising and forward locomotion, in the open half of the platform, which decreasing the frequency of stationary elements, such as sitting still and inactivity, in the enclosed half of the platform.

b) The Elevated Plus-maze Paradigm in mice

This test is basically as described in Behav. Pharmacol., 1998, 8: 477-496.

At doses of about 1 to about 10 mg/kg p.o., the agents of the invention significantly increase the time spent on the open arms. These findings are consistent with the Half Enclosed Platform test results.

c) The amphetamine-induced hypermotility test

This test is performed according to the method described by Arnt J in Eur. J. Pharmacol, 283, 55-62 (1995).

At doses of about 0.01 to about 10 mg/kg s.c., the agents of the invention significantly inhibit the amphetamine-induced locomotion in the animals.

In view of their behavioral desinhibitory (=anxiolytic or antidepressant-like) and sociotropic activity, the agents of the invention are useful in the treatment of affective disorders including bipolar disorders, e.g. manic and depressive disorders, cyclothymia, schizo-affective disorders and excessive mood swings where behavioral stabilization is desired. In addition, the compounds are indicated in ADHD (attention deficit hyperactivity disorders) and behavioral disorders associated with dementia and Parkinson's disease. As evidenced by the elevated maze test, an effect is anticipated In anxiety disorders, (e.g. generalized anxiety, social phobia and agoraphobia), as well as those behavioral states characterized by social withdrawal (e.g. autism and psychoses with predominant negative symptoms [hebephrenia]).

For the above-mentioned indications the appropriate dosage will vary depending upon, for example, the compound employed, the host, the mode of administration and the nature and severity of the condition being treated. However, in general, satisfactory results in animals are indicated to be obtained at a daily dosage of from about 1 to about 50 mg/kg animal body weight. Daily doses in larger mammals, such as humans, depend on the outcome of clinical studies in the different behavioral disorders and vary from about 1 to about 50 mg of an agent of the invention, conveniently administered in divided doses up to two times a day.

The agents of the invention may be administered in any usual manner, e.g. orally, for example in the form of tablets or capsules, or parenterally, for example in the form of injection solutions or suspensions.

The present invention also provides pharmaceutical compositions comprising an agent of the invention in association with at least one pharmaceutical carrier or diluent, for use in the treatment of affective and attention disorders. Such compositions may be manufactured in conventional manner. Unit dosage forms may contain for example from about 0.1 mg to about 25 mg of the compound of formula I.

The invention further provides the use of an agent of the invention for the manufacture of a pharmaceutical composition for the treatment of affective and attention/behavioral disorders.

The invention furthermore provides a method for the treatment of affective and attention disorders in a subject in need of such treatment, which comprises administering to said subject a therapeutically effective amount of an agent of the invention.

1. The use 1-[4-[3-[4-(6-fluoro-1,2-benzisoxazol-3-yl)-1-piperidinyl]propoxy]-3-methoxyphenyl]ethanone and its pharmaceutically acceptable acid addition salts, for the treatment of affective and attention/behavioral disorders.

2. A pharmaceutical composition comprising 1-[4-[3-[4-(6-fluoro-1,2-benzisoxazol-3-yl)-1-piperidinyl]propoxy]-3-methoxyphenyl]ethanone or a pharmaceutically acceptable acid addition salt thereof, in association with at least one pharmaceutical carrier or diluent, for use in the treatment of affective and attention/behavioral disorders.

3. The use of 1-[4-[3-[4-(6-fluoro-1,2-benzisoxazol-3-yl)-1-piperidinyl]propoxy]-3-methoxyphenyl]ethanone or a pharmaceutically acceptable acid addition salt thereof, for the manufacture of a pharmaceutical composition for the treatment of affective and attention/behavioral disorders.

4. A method for the treatment of affective and attention/behavioral disorders in a subject in need of such treatment, which comprises administering to said subject a therapeutically effective amount of 1-[4-[3-[4-(6-fluoro-1,2-benzisoxazol-3-yl)-1-piperidinyl]propoxy]-3-methoxyphenyl]ethanone or a pharmaceutically acceptable acid addition salt thereof.
